(19) United States
(12) Patent Application Publication
(10) Pub. No.: US 2014/0134221 A1
(43) Pub. Date: May 15, 2014

(54) AGENT FOR PROVIDING POSITIVE SURFACE CHARGE ON UV PROTECTION VESICLES

(71) Applicants: Monika Beyer, Nidderau-Ostheim (DE); Michael Mandred Sacher, Schluchtern-Breitenbach (DE); Dirk Teichmüller, Linsengericht-Geislitz (DE); Sarah Teichmüller, Linsengericht-Geislitz (DE)

(72) Inventors: Monika Beyer, Nidderau-Ostheim (DE); Michael Mandred Sacher, Schluchtern-Breitenbach (DE); Dirk Teichmüller, Linsengericht-Geislitz (DE); Sarah Teichmüller, Linsengericht-Geislitz (DE)

(73) Assignee: AIR PRODUCTS SCHLÜCHTERN GMBH, Schlüchtern (DE)

(21) Appl. No.: 14/118,378

(22) PCT Filed: Feb. 24, 2012
(86) PCT No.: PCT/EP2012/053136

§ 371 Date: Jan. 10, 2014

(30) Foreign Application Priority Data
May 19, 2011 (DE) 10 2011 076 149.7

(51) Int. Cl. A61K 8/11

(52) U.S. Cl. CPC A61K 8/11
USPC 424/401; 424/59; 424/60

(57) ABSTRACT

In order to prepare a UV protective agent for topical application to the skin or hair with very high UV filter substance concentration and with good adhesion to the skin and hair, according to the invention the UV protective agent is proposed with at least one UV filter substance, which is encapsulated in a vesicular carrier system, in which the UV protective agent is characterized by the fact that the at least one UV filter substance encapsulated in the vesicular carrier system is lipophilic and the vesicular carrier system consists of vesicles constructed from hydrophobized polysaccharides and having a particle size from 10 to 1000 nm, as well as a positive surface charge with a zeta potential in the range from 1 to 150 mV by means of the charge donors contained therein. In addition, use of such a UV protective agent in corresponding cosmetic and/or pharmaceutical formulations is proposed.

The present invention concerns a UV protective agent for topical application to the skin or hair with at least one lipophilic UV filter substance, which is encapsulated in a vesicular carrier system of hydrophobized polysaccharides that does not penetrate the skin or hair, the vesicles of the carrier system having a positive surface charge. Moreover, the present invention concerns use of such a UV protective agent in corresponding cosmetic and/or pharmaceutical formulations.

The sunlight visible to the human eye extends over a radiation spectrum from 400 to 800 nm. The spectrum of UV radiation (UV=ultraviolet) lies below 400 nm. Although ultraviolet radiation represents the lowest energy ionizing form of radiation, it can be harmful for body surfaces exposed to this radiation.

UVA rays (320 to 400 nm) produce, among other things, damage to collagens so that skin loses its tensile force and can therefore age prematurely. Increased radiation load in the UVA range also leads to higher risk of melanoma. UVB rays (280 to 320 nm) lead to skin reddening related to inflammation and cause the universally known sunburn.

A photochemically-induced loss of proteins and degradation of the hair dye melanine accompanies UV radiation for hair. UVB rays are then mostly responsible for morphological damage to hair, like degradation of hair proteins, whereas UVA rays in particular can cause biochemical changes and color changes. Amino acids, from which hair proteins are composed, are light-sensitive. Their photochemical damage produces free radicals, which in turn further damage the protein structure of keratin and therefore hair.

Consequently, there is a demand for UV protective agents to protect against the aforementioned damaging effects of the UV fraction in sunlight. Such UV protective agents are ordinarily applied to the skin or hair to prevent the negative effects of solar radiation (like sunburn, skin aging, hair damage). For this purpose the mentioned UV protective agents contain appropriate UV filter substances.

A distinction is made among the UV filter substances between chemical and physical UV filter substances. Chemical filter substances absorb high-energy radiation and release it again as low-energy longer wave radiation or heat. Physical filter substances, on the other hand, mostly scatter and reflect the light. Since the individual substances generally offer no protection over the entire UV spectrum, several substances are generally combined.

In order to achieve optimal UV protection the highest possible UV filter substance concentration must be present in a UV protective agent. This is only satisfactorily guaranteed to the desired extent conditionally in many ordinary UV protective agents. In particular, the concentrations desired for optimal UV protection are often not reached if a combination of two or more different UV filter substances with synergistic effect spectrum in high concentration is required in order to achieve a UV protective agent with an optimal broadband protection over different areas of the relevant UV spectrum.

The underlying task of the invention is therefore to furnish a UV protective agent in which the UV filter substance can be contained in very high concentrations. In particular, it is to be possible to stably incorporate two or more different UV filter substances with synergistic effect spectrum both in very high concentration in the UV protective agent in order to achieve a UV protective agent with optimal broadband protection over different areas of the relevant UV spectrum. In addition, there should be a possibility of stably integrating so-called photostabilizers together with the UV filter substances in the UV protective agent, which are capable of protecting UV filter substances from decomposition by UV radiation and therefore stabilizing them.

It is also desired that the UV protective agent with the aforementioned capabilities also offers good adhesion to skin and hair in order to guarantee the longest possible residence time of the UV filter substance or UV filter substances on the skin or hair. In particular, adhesion should be effective enough that the UV filter substances or UV filter substances can also withstand several washing processes on the skin or hair.

This task is solved according to the invention by a UV protective agent of the type just mentioned in which the vesicles from hydrophobized polysaccharides have a particle size from 10 to 1000 nm and a positive surface charge with a zeta potential in the range from 1 to 150 mV and in which the positive surface charge is produced by the fact that the vesicles have positively charged molecules as charge donors in addition to the polysaccharides from which the vesicles are constructed, these charge donors being selected from one or more of the following charge donors a) to f):

a) N-Stearyl-N,N-dimethyl-N-(2-hydroxy-3-panthenyl)propylammonium Salts with the Formula (I)

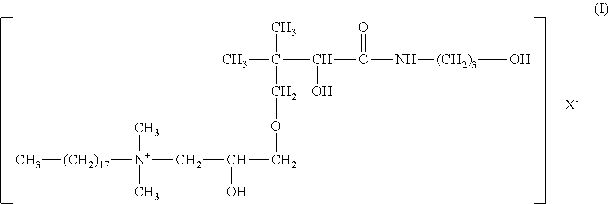

in which X⁻ in the aforementioned formula (I) is a cosmetically or pharmaceutically compatible organic or inorganic anion. X⁻ is preferably a halide ion or the anion of an organic acid chosen among a cosmetically or pharmaceutically compatible carboxylic acid or sulfonic acid. With particular preference X⁻ is bromide, chloride, fluoride, iodide, saccharinate, tosylate or methosulfate, with particular preference chloride.

b) Positively Charged Quaternary Sugar Derivatives (Sugar Quats) with the Formula (II)

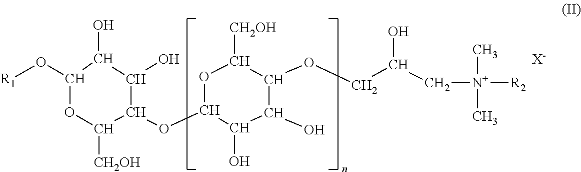

The quaternary sugar derivatives (sugar quats) preferably have monosaccharide units with 6 carbon atoms. The sugar quats with particular preference are chosen among glucose, fructose, mannose and/or galactose. In particularly preferred variants monosaccharides [form] disaccharide units. Sugar quats in which the monosaccharides form the disaccharide units maltose and/or lactose are particularly preferred.
The quaternary sugar derivatives are preferably ether compounds of alkyl glucopyranosides (n=0) and/or alkyl glucopyranosylglucopyranosides (n=1) with N-alkyl-N,N-dimethyl-N-(2,3-dihydroxypropyl)ammonium salts and their combinations. The alkyl group R₁ bonded to the sugar unit then preferably has an unbranched or branched alkyl chain with C₄-C₁₄, especially C₄, C₁₀ and/or C₁₂ carbon atoms. The alkyl group R₂ bonded to the quaternary nitrogen atom is preferably a methyl group or with particular preference as an unbranched or branched alkyl chain with C₄-C₁₈ and especially C₄, C₁₂ and/or C₁₈ carbon atoms.
The aforementioned ether compounds can be uniform substances. However, mostly natural oils are generally used to produce these substances, preferably coconut oil and palm oil/palm kernel oil, so that in addition to the primarily mentioned unbranched or branched alkyl groups R₁ and R₂, additional alkyl and alkenyl groups of other chain lengths can be present in subordinate amounts in the substance mixture according to the chosen starting material.
The quaternary sugar derivatives with formula (II) also explicitly include oligosaccharide compounds in which the number of monosaccharides units n is preferably 1<n<100, R₁ preferably represents a hydrogen atom and R₂ preferably an alkyl group with C₈-C₂₀ and especially C₁₈ carbon atoms.
The anion X⁻ in the aforementioned formula (II) is preferably a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate, with particular preference chloride.

c) Positively Charged Phospholipids Consisting of Diester and Triester Phosphatides with the Formula (III)

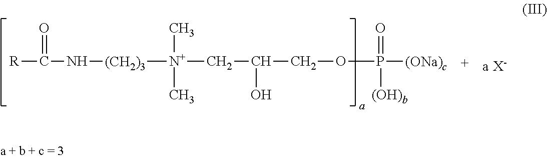

The positively charged phospholipids and their salts with formula (III) preferably have an unbranched or branched alkyl group and/or alkenyl group R with C₇-C₂₅ and especially C₁₃-C₂₁ carbon atoms. With particular preference the alkyl group R has C₁₃, C₁₇ and/or C₂₁ carbon atoms. In the case of an alkenyl group R with particular preference has chains with C₁₇ carbon atoms. One, two or three double bonds then preferably lie in the alkenyl group.
Mostly natural oils are used to produce these substances, preferably coconut oil, palm oil/palm kernel oil, sunflower oil, thistle oil, olive oil, grapeseed oil, borage oil and castor oil, etc. so that in addition to the primarily mentioned unbranched or branched alkyl and/or alkenyl groups R, additional alkenyl and alkyl groups of other chain lengths can be present in subordinate amounts of the substance mixture according to the chosen starting material. In the case of castor oil the C₁₇ alkenyl group has a functional hydroxyl group.

The anion X⁻ in the aforementioned formula is preferably a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate, with particular preference chloride.

d) Positively Charged N-(3-alkylamido)propyl-N,N-dimethyl-N-(2,3-dihydroxypropyl)-Ammonium Salts or N-(3-alkenylamido)propyl-N,N-dimethyl-N-(2,3-dihydroxypropyl)-Ammonium Salts with Formula (IV)

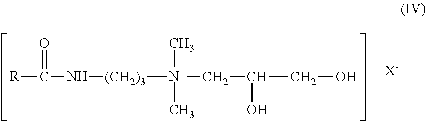

or N-(3-ricinylamido)propyl-N,N-dimethyl-N-(2,3-dihydroxypropyl)ammonium salts.
The positively charged N-(3-alkylamido)propyl-N,N-dimethyl-N-(2,3-dihydroxypropyl)-ammonium salts of formula (IV) preferably have an unbranched or branched alkyl group R with C₇-C₂₅ and especially C₁₃-C₂₁ carbon atoms. With particular preference the alkyl group has C₁₃, C₁₇ and/or C₂₁ carbon atoms. The N-(3-alkylamido)propyl-N,N-dimethyl--N-(2,3-dihydroxypropyl)ammonium salts with formula (IV) can be uniform substances, but generally natural oil are mostly used to produce these substances, preferably coconut oil and palm oil/palm kernel oil, but optionally also tallow so that, in addition to the primarily mentioned unbranched or branched alkyl groups R, additional alkyl and alkenyl groups of other chain lengths can be present in subordinate amounts in the substance mixture according to the chosen starting material.
The anion X⁻ in the aforementioned formula is preferably a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate, with particular preference chloride.
The N-(3-alkenylamido)propyl-N,N-dimethyl-N-(2,3-dihydroxypropyl)ammonium salts with formula (IV) preferably have an unbranched or branched alkenyl group R with C₁₃-C₂₃ and especially C₁₇ carbon atoms. One, two or three double bonds are then preferably present in the alkenyl group.
The N-(3-alkenylamido)propyl-N,N-dimethyl-N-(2,3-dihydroxypropyl)ammonium salts with formula (IV) can be uniform substances but generally natural oils are mostly used to produce these substances, preferably sunflower oil, thistle oil, olive oil, grapeseed oil, borage oil and castor oil, etc. so that, in addition to the primarily mentioned unbranched or branched alkenyl groups R, additional alkenyl and alkyl groups of other chain lengths can be present in subordinate amounts in the substance mixture according to the chosen starting material.
The anion X⁻ in the aforementioned formula is preferably a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate, with particular preference chloride.

e) Positively Charged Quaternary N,N,N-trimethyl-N-(2-hydroxy-3-“R ether”-propyl)-Ammonium Salts With Formula (V)

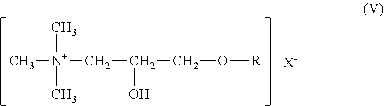

The quaternary N,N,N-trimethyl-N-(2-hydroxy-3-“R ether”-propyl)ammonium salts with formula (V) preferably have a group R which originates from natural sources. R with particular preference represents honey, hyaluronic acid, the polysaccharides xanthan gum and trehalose as well as hydrolyzates of silk, wheat starch, corn starch, keratin and protein hydrolyzates from wheat, rice, soya, oats, etc.
The anion X⁻ in the aforementioned formula is preferably a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate.
As an alternative the vesicles, in addition to the charge donors mentioned above, can have one or more of the following additional charge donors:

f) Positively Charged Quaternary C₁₂-C₂₂ Alkyltrimethylammonium Salts (or Fatty Acid Trimonium Salts) With Formula (VI)

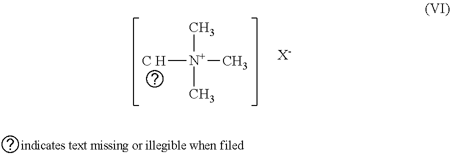

The quaternary alkyltrimethylammonium salts (or fatty acid trimonium salts) with formula (VI) preferably an alkyl group with C₁₂-C₂₂ carbon atoms. n in the aforementioned alkyltrimethylammonium salt formula is preferably equal to 22.
X⁻ in the aforementioned formula (VI) is a halide ion or the anion of an organic acid chosen among cosmetically or pharmaceutically compatible carboxylic acid or sulfonic acid. With particular preference X⁻ is bromide, chloride, fluoride, iodide, saccharinate, tosylate or methosulfate.

It could be demonstrated that the charge donors according to the invention offer the advantage that large amounts of lipophilic UV filter substance can be stably incorporated in a nanoscale novel carrier system and thus optimize the effect of the UV protective agent.

A further advantage is that through the charge donors of the present invention adhesion of the vesicles to skin and hair and therefore the residence time of the UV filter substance can be significantly increased. The preparation according to the invention demonstrably exhibits in this respect good film formation on skin and hair, as well as effective adhesion surviving several washings. Both the amount of UV filter substance remaining on the skin and hair after several washings and its protective effect could be significantly increased by the preparation according to the invention relative to free UV filter substances.

On the other hand, incorporation in high and effective concentration in hydrophilic cosmetic and/or pharmaceutical formulations is made possible by encapsulation of the UV filter substance. A combination of several oil-soluble UV filter substances with a synergistic effect spectrum in a carrier system is also possible through a UV protective agent with the properties according to the invention. In this way an extremely effective broadband protection can be achieve over the UVA and UVB range.

The term “encapsulation” includes in conjunction with the present invention essential embedding of UV filter substances between the polysaccharide molecules and the structures formed by the polysaccharide molecules as well as inclusion of UV filter substances in the vesicle interior.

The lipid vesicles according to the invention have a zeta potential in the range from 1 to 150 mV. The term “zeta potential” describes the electric potential of a shear layer of a moving particle in a suspension. Measurement of the zeta potential can occur by moving particles through an applied electric field. The zeta potential can then be calculated from the resulting velocity in the particles. Preferred lipid vesicles have a zeta potential from 30 to 100 mV. In particularly preferred lipid vesicles the zeta potential is 40 to 60 mV.

The zeta potential in conjunction with the present invention is determined by means of laser Doppler electrophoresis. The measurements then occur in strongly diluted aqueous salt solutions, in which case samples being measured are ordinarily present in concentrations of 0.01-0.1 wt % in 1 mM sodium chloride solution. The pH values of the sample solutions lie in the pH value specification range of the corresponding product being measured (UV protective agent and formulations according to the invention) in which product-specific pH values in the range from pH 5.5 to maximum pH 7.5 are generally encountered.

The improved adhesion of the UV protective agent is based on the positive charge of the vessel surface. The positive charge of the vessel surface leads to improved adherence of the vesicles to the surface of skin cells. Moreover, the positive charge of the vesicles also leads to strong adhesion to the hair surface. It has also been shown that this improved adhesion is also connected with the nanoscale particle size of the carrier vesicles according to the invention, in which all these findings should be related but not in binding fashion to the present invention and therefore should also not restrict the scope of the invention.

The particle size of the lipid vesicles according to the invention is in the range from 10 to 1000 nm. In certain variants the particle size is 100 to 400 nm. In other variants it is 100 to 350 nm. The particle size is preferred variants is 100 to 250 nm.

In conjunction with the present invention the term “particle size” means the average particle size. The average particle size can be determined by photon correlation spectroscopy. Photon correlation spectroscopy, also referred to as dynamic light scattering, is an optical measurement method for determination of the size distribution of vesicles and particles in liquids. The method utilizes scattering of laser light by the vesicles.

Through the combination according to the invention of positive charge of the vesicle surface and nanoscale particle size of the carrier vesicles formed from hydrophobized polysaccharides, a situation is achieved in skin and hair applications in which the UV filter substances contained in the vesicles remain permanently on the surfaces being protected. However, these vesicles do not penetrate the skin or hair. This means the vesicles do not enter the skin or hair, which can be demonstrated by means of fluorescence microscopy with a carrier system loaded with a fluorescence dye.

In the present invention one or more of the aforementioned positively-charged charge donors is preferably used so that the charge donor percentage in the vesicles lies in the range from 0.01 to 10 wt % referred to the total weight of the vesicles. In certain variants the charge donor fraction lies in the range from 0.01 to 2.0 wt %.

The fractions stated in wt % refer essentially to the entire formulation of the UV protective agent according to formula stipulations, including the water fraction required for vesicle formation, unless otherwise stated.

The stated fraction also includes all substances that clearly fall under the definition of the respective substance group. Consequently the stated charge donor fraction in the variants with a charge donor includes the fraction of this charge donor and in variants with several charge donors the sum of all charge donors. This also equally applies for fractions of hydrophobized polysaccharide, filter substance and auxiliary or other substance or substance fractions stated in conjunction with this invention.

The polysaccharide framework of the hydrophobized polysaccharides that can be used in the UV protective agent for formation of vesicles can be chosen among all cosmetically or pharmaceutically compatible polysaccharides that are capable of forming vesicles. They are preferably water-soluble polysaccharides and/or their ethers with short-chain alcohols (C₁ to C₄), in which the water-soluble polysaccharides can be linear, branched, comb-like and/or stellate. Linear water-soluble polysaccharides are particularly preferred. Copolymers or block copolymers of different monosaccharide units and/or monosaccharide units linked to each other in different ways are also considered.

The hydrophobized polysaccharides from which the vesicles are constructed preferably have a polysaccharide framework for polyglucose or polyfructose. Preferred hydrophobized polysaccharides with a polysaccharide framework from a polyglucose are cellulose, methyl cellulose, hydroxyethyl cellulose, amylose, amylopectin and dextrin. A preferred hydrophobized polysaccharide with polysaccharide framework from polyfructose is inulin.

The hydrophobized polysaccharide in the present invention preferably includes on average from 5 to 1000 monosaccharide units. With further preference it has 10 to 500 monosaccharide units. With particular preference there are 20 to 100 monosaccharide units.

In special variants mixtures of the aforementioned polysaccharides can also be used with the stipulation that these mixtures are capable of forming vesicles.

The fraction of polysaccharides in the vesicles, referred to the total weight of the vesicles, lies in the range from 1 to 85 wt %. The polysaccharide fraction referred to the total weight of the vesicles is preferably 5 to 25 wt %. With particular preference the polysaccharide fraction is 8 to 15 wt %.

In preferred variants the hydrophobized polysaccharide from which the vesicles are constructed are hydrophobized by the fact that they have a polysaccharide framework with C₃-C₂₂ alkyl groups, which are bonded to the hydroxy group of the polysaccharide via alkyl ether bonds or via alkyl urethane bonds or which are bonded to the polysaccharide framework via a linker (for example, polyether linker, polyethylene glycol linker).

The molecular weight of the hydrophobically modified polysaccharide in preferred variants lies in the range from 5000 to 500,000 g/mol. The range from 5000 to 100,000 g/mol is preferred here.

In preferred hydrophobically modified polysaccharides the ratio from the number of hydrophobically modified groups and modifiable groups (degree of modification) is from 0.01 to 0.9. The degree of modification in particular preferred variants is from 0.03 to 0.15.

The hydrophobic groups and the polysaccharide backbone in certain variants can be mono- or poly-substituted with halogen, hydroxy, alkoxy, amino, alkylamino, aryl, arylalkyl, carboxy, carboxy ester and cycloaliphatic groups. However, the hydrophobically modified polysaccharides are preferably nonionic compounds.

Particularly preferred hydrophobically-modified polysaccharides according to the invention are inulin lauryl carbamates, cetyl hydroxyethyl cellulose, hydroxy-C₃₋₆-alkyl-modified cellulose, especially hydroxypropyl cellulose and hydroxypropylmethyl cellulose.

All chemically or physically UV-filtering substances, provided they are lipophilic are considered without restriction as UV filter substances in the present invention.

A UV filter substance according to present invention is lipophilic, if it has an n-octanol-water partition coefficient K_{ow} greater than 1 at 25° C.

The chemical filter substances absorb high-energy radiation and release it as lower energy, longer wave radiation or heat. The physical filter substances scatter and reflect the light mostly.

Preferred examples for UV filter substances that can be used in the present invention are without restriction to this 3-benzylidene camphor, 4-methylbenzylidene camphor, 1-benzophenone, 2-benzophenone, 3-benzophenone, 4-benzophenone, 5-benzophenone, 6-benzophenone, 9-benzophenone, benzylidene camphor sulfonic acid, bis-ethylhexyloxyphenolmethoxyphenyl triazine, butylmethoxydibenzoyl methane, camphor benzalkonium methosulfate, diethylaminohydroxybenzoylhexylbenzoate, diethylhexylbutramidotriazone, disodium phenyldibenzimidazole tetrasulfonate, drometrizole trisiloxane, ethylhexyldimethyl-PABA, ethylhexylmethoxycinnamate, ethylhexylsalicylate, ethylhexyltriazone, homosalate, isoamyl-p-methoxycinnamate, methylene-bis-benzotriazolyltetramethylbutyl phenol, octo-crylene, PEG-25-PABA, phenyl benzimidazole sulfonic acid, polyacrylamidomethylbenzylidene camphor, polysilicone 15, potassium phenylbenzimidazole sulfonate, sodium mangoseedate, sodium phenylbenzimidazole sulfonate, TEA-phenylbenzimidazole sulfonate, terephthalylidene dicamphor sulfonic acid, ferulic acid, cinoxate, diisopropylmethylcinnamate, 4-(2-β-glucopyranosiloxy)propoxy-2-hydroxybenzophenone, glyceryl ethylhexanoate dimethoxycinnamate, isopentyl trimethoxycinnamate trisiloxane.

The fraction of UV filter substance in the present invention is preferably 1 to 65 wt % referred to the total weight of the complete formulation of the UV protective agent according to formula stipulations, including the water fraction required for vesicle formation.

Since the individual filter substances generally do not offer protection over the entire UV spectrum, several filter substances are combined in preferred variance of the invention in order to achieve the broadest possible UV protection.

The UV protective agent according to the invention is used in cosmetic and/or pharmaceutical formulations, pharmaceutical formulations being those that fall under the drug law.

The formulations can find application as auxiliaries and additives that are ordinarily used in cosmetic or pharmaceutical preparations. In particular, under the term “auxiliary” in conjunction with the present invention those additives fall which have an effect on the physical properties of the vesicles and their stability and/or serve to preserve the UV protective agents. Examples of such auxiliaries are oils, alcohols, polyols, antioxidants, gel-forming agents, buffers, preservatives, bactericides and germ inhibitors, consistency agents, thickeners and sequestering agents.

UV protective agent according to the present invention is preferably characterized by the fact that the auxiliary fraction referred to the total weight of the entire formulation of the UV protective agent according to formula stipulations, including the water fraction required for vesicle formation lies in the range from 0.01 to 10 wt %.

In a specific variant of the present invention the formulation includes with the UV protective agent according to the invention also a liposomal carrier system that penetrates the skin or hair in which at least one antioxidant active ingredient is encapsulated. The antioxidant active ingredient prevents or inhibits chemically or photochemically induced oxidation of sensitive molecules, but the formula component can also be an ingredient of a skin-physiological or hair-physiological system in which it terminates radical chain reactions as a radical scavenger or is more easily and quickly oxidized as a reducing substance with very limited intrinsic redox potential than the molecules for systems being protected or supports in synergistic fashion the effect of other antioxidants, in which case it deactivates protagonists of the oxidative process, for example, by chelate formation.

In preparation of the formulation with the UV protective agent according to the invention in a specific variant a liposomal carrier system that penetrates the skin or hair is therefore additionally used, in which at least one antioxidant active ingredient is encapsulated.

The aforementioned liposomal carrier system preferably consists of vesicles constructed from lipids. The vesicles with particular preference have a particle size from 10 to 1000 nm.

The lipids from which the liposomal vesicles are constructed are preferably chosen among ceramides, phospholipids, glycosphingolipids and/or diacylglycosides.

The fraction of lipids from which the liposomal vesicles are constructed referred to the entire formulation as preferably 1 to 20 wt %.

The at least antioxidant active ingredient encapsulated in the liposomal vesicles is preferably chosen among lipophilic and hydrophilic antioxidants, which were isolated from natural sources or produced chemically or by biotechnology or their combinations. With particular preference the at least one antioxidant active ingredient (without restriction to this) is chosen from the group of vitamins, preferably ascorbic acid (vitamin C), its salts, its 2-0 and/or 3-O ether compounds and with particular preference 3O-ethylascorbic acid, as well as mono-, di- and tetraesters of ascorbic acid with palmitic acid, stearic acid, isostearic acid, linoleic acid, sulfuric acid and phosphoric acid as well as their alkali and alkaline earth salts and ascorbyl-2-O-glucoside, tocopherol (vitamin E), its esters with acetic acid, ferulic acid, linoleic acid, oleic acid, nicotinic acid, retinoic acids, succinic acid, maleic acid and phosphoric acid as well as their alkali and alkaline earth salts, tocotrienol and tocopheryl-6-O-D-glucopyranoside.

As an alternative the at least one antioxidant active ingredient (without restriction to this) can be chosen from the group of cosmetically or pharmaceutically compatible phenol compounds with one or more hydroxyl groups, preferably tert-butyl-4-methoxyphenol (BHA), 2,6-ditert-butyl-p-cresol (BHT) and derivatives of resorcinol, for example, 4-butylresorcinol, 4-(1-phenyethyl)resorcinol, etc. and cosmetically or pharmaceutically compatible derivatives of hydroquinone, for example, ubiquinol, its oxidized quinoid form 6-all-trans-decaprenyl-2,3-dimethoxy-5-methyl-1,4-benzoquinone (coenzyme Q10) and its alkali salts, 6-(10-hydroxy-decyl)-2,3-dimethoxy-5-methyl-1,4-benzoquinone (idebenone) and its linoleic acid esters as well as hydroxyl group-containing benzoic acid derivatives (for example, vanillic acid, gallic acid, protocatechuic acid, etc.) and cinnamic acid derivatives (for example, caffeic acid, p-coumaric acid, etc.) as well as their esters with C₂-C₁₈ alcohols or fatty alcohols.

The at least one antioxidant active ingredient (without restriction for this) can also be chosen from the group of hydroxyl group-containing stilbene derivatives (for example, resveratrol) as well as ellagic acid and rosmarinic acid.

The at least one antioxidant active ingredient (without restriction for this) with particular preference is chosen from the group of polyphenol compounds, in which case the subgroups of a chemical structural framework of chalcones, flavones (for example, luteolin), flavonols (for example, quercetin, rutin, etc.), flavanols (catechol, gallocatechol, epicatechol, epigallocatechol gallate, dimeric and trimeric catechols OPC, tannins, etc.), flavanones (for example, hesperetin), flavanonols, isoflavones (for example, genistein, licoricidin, etc.), anthocyans and aurones (as well as mostly the glycosides of the aforementioned compound groups).

Since the aforementioned polyphenol compounds are secondary plant substances, the at least one antioxidant active ingredient (without restriction to this) with particular preference is an aqueous, alcoholic, hydroglycol, oil and/or CO₂ extract of polyphenol-rich plant and plant parts, in which case roots, stems, leaves, flowers, fruits, fruit husks and/or seeds can serve as extraction base, depending on the plant species. The extract can be present as liquid, concentrated and/or also as a solid after spray or freeze drying. These extracts then naturally generally contain no isolated uniform substance but instead substance mixtures of the aforementioned compounds in variable composition.

With particular preference the polyphenol-rich plant extracts are chosen among rosemary extracts, ginger extracts, thyme extracts, sage extracts, tea, mostly green tea extracts, grapeseed and grape husk extracts, chokeberry extracts, pomegranate extracts, rooibos extracts, nut gall extracts, hops extracts, ginkgo extracts, Melissa extracts, etc.

In other alternative variants of the invention the at least one antioxidant of active ingredient is chosen from the group of antioxidant enzymes, preferably superoxide dismutase (SOD), glutathione peroxidase (GPX) and catalase. In another alternative variant of the invention the at least one antioxidant active ingredient is chosen from the group of sulfides and sulfites, preferably glutathione and alkali and alkaline earth sulfites and disulfites and alkali bisulfites.

The fraction of the at least one antioxidant active ingredient referred to the entire formulation is preferably 0.01 to 20 wt %.

In the variants of the invention with additional liposomal carrier system the formulation preferably additionally has one or more auxiliaries that are preferably chosen among: sequestering agents, like ethylenediamine tetraacetic acid (EDTA) and/or its salts, nitrilotriacetic acid (NTA) and/or its salts, cosmetically or pharmaceutically compatible phosphates and/or phosphonates, salts and esters of oxalic acid and/or tartaric acid as well as with particular preferences N,N-bis(carboxymethyl)glutamic acid and/or its salts, etc., since the effect of other antioxidants is supported by them in which protagonists of the oxidative processes are deactivated by chelate formation.

The UV protective agents according to invention are preferably used for cosmetic and/or pharmaceutical formulations that are suitable for topical application. The UV protective agents according to the invention can be present for this purpose in all formulations appropriate for topical application, for example, in the form of a gel, a cream, an ointment, a spray, a lotion, an aqueous or aqueous-alcoholic preparation. For this purpose the UV protective agent according to the invention can be incorporated in a carrier matrix. The carrier matrix can be gel formulations, cream formulations, lotions, mask applications, tints and wax pencil formulations, etc.

For applications in the skin area the UV protective agent according to the invention is preferably applied in a lotion, a cream, an ointment, a gel, an aqueous fluid, a face lotion, a sun product or mask.

For applications in the hair area the UV protective agent according to the invention is preferably applied in a shampoo (preferably with mild surfactants), a rinse, a hair gel, a conditioner, a hair tonic, a hair styling product or a hair care product.

Naturally all the components of the UV protective agent according to the invention and its formulations are pharmaceutically, cosmetically or dermatologically compatible substances. A substance according to this invention is pharmaceutically, cosmetically or dermatologically compatible, if it is not toxic and can be applied topically in most potential applications without leading to undesired physiological reactions, for example, reddening or formation of itching spontaneously or after some time in the user.

For purposes of original disclosure it is pointed out that all features that follow from the present description and the claims to one skilled in the art, even if they were specifically described only in conjunction with specific other features, can be combined both individually and in any combination with other features or feature groups disclosed here, if this was not explicitly ruled out or chemical, physical-chemical, cosmetic, pharmacological or dermatological circumstances makes such combinations impossible or unreasonable. Comprehensive explicit presentation of all conceivable feature combinations is dispensed with here in the interest of brevity and readability of the description.

1. UV protective agent for topical application to the skin or hair with at least one UV filter substance, which is encapsulated in a vesicular carrier system that does not penetrate the skin or hair, in which the at least one UV filter substance is lipophilic and the vesicular carrier system consists of vesicles constructed from hydrophobized polysaccharides having a particle size from 10 to 1000 nm, as well as a positive surface charge with a zeta potential in the range from 1 to 150 mV, characterized by the fact that the positive surface charge is produced by the fact that the vesicles have positively charged molecules as charge donors, in addition to the polysaccharides from which the vesicles are constructed, these charge donors being chosen among:
a) N-stearyl-N,N-dimethyl-N-(2-hydroxy-3-panthenyl)propylammonium salts with the formula (I), 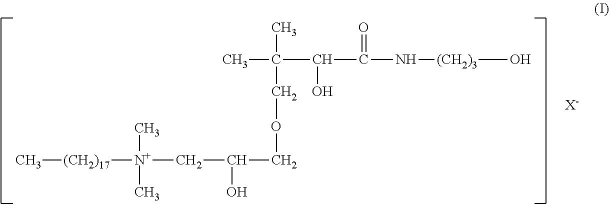
b) positively charged quaternary sugar derivatives with the formula (II) 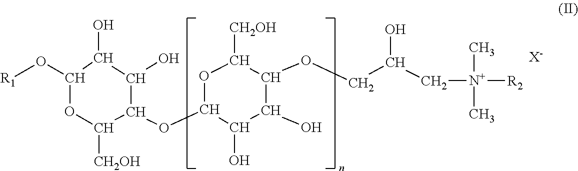
c) positively charged phospholipids chosen among diester and triester phosphatides with the formula (III) 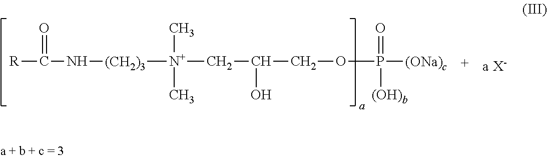
d) positively charged N-(3-alkylamido)propyl-N,N-dimethyl-N-(2,3-dihydroxy-propyl)ammonium salts and N-(3-alkenylamido)propyl-N,N-dimethyl-N-(2,3-dihydroxypropyl)ammonium salts with the formula (IV) 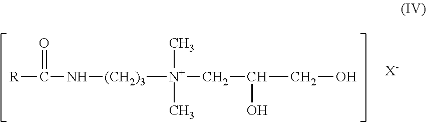
as well as N-(3-ricinylamido)propyl-N,N-dimethyl-N-(2,3-dihydroxypropyl)-ammonium salts,
e) positively charged quaternary N,N,N-trimethyl-N-(2-hydroxy-3-“R ether”-propyl)ammonium salts with the formula (V) 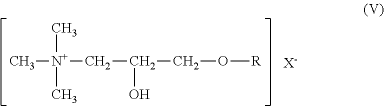
or their combinations,
and in which the vesicles for formation of the positive surface charge alternately have in addition to the aforementioned charge donors
f) one or more positively charged quaternary C₁₂-C₂₂ alkyltrimethylammonium salts (or fatty acid trimonium salts) with formula (VI) 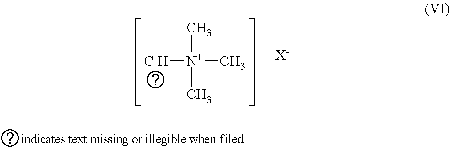
in which X⁻ in the above formulas (I) to (VI) is a cosmetically or pharmaceutically compatible organic or inorganic anion.

2. UV protective agent according to claim 1, characterized by the fact that the quaternary sugar derivatives of formula (II) are formed from sugar units chosen among glucose, fructose, mannose, galactose, maltose, lactose, ether compounds of alkyl glucopyranosides (n=0) and/or alkyl glucopyranosylglucopyranosides (n=1) with N-alkyl-N,N-dimethyl-N-(2,3-dihydroxypropyl)ammonium salts and their combinations.

3. UV protective agent according to claim 1, characterized by the fact that the positively charged phospholipids and their salts of formula (III) have an unbranched or branched alkyl group and/or alkenyl group R with C₇-C₂₅.

4. UV protective agent according to claim 1, characterized by the fact that the positively charged N-(3 -alkylamido)propyl-N,N-dimethyl-N-(2,3 -dihydroxypropyl)-ammonium salts with formula (IV) have an unbranched or branched alkyl group R with C₇-C₂₅ and the N-(3 -alkenylamido)propyl-N,N-dimethyl-N-(2,3 -dihydroxypropyl)-ammonium salts with formula (IV) have an unbranched or branched alkenyl group R with C₁₃-C₂₃.

5. UV protective agent according to claim 1, characterized by the fact that the quaternary alkyl trimethylammonium salts have an alkyl group with C₁₂⁻C₂₂ carbon atoms.

6. UV protective agent according to claim 1, characterized by the fact that the quaternary N,N,N-trimethyl-N-(2-hydroxy-3-“R ether”-propyl)ammonium salts with formula (V) have a group R, which represents honey, hyaluronic acid, the polysaccharides xanthan gum and trehalose as well as hydrolyzates of silk, wheat starch, corn starch, keratin and protein hydrolyzates from wheat, rice, soya, oats.

7. UV protective agent according to claim 1, characterized by the fact that X⁻ is chosen among a cosmetically or pharmaceutically compatible carboxylic acid, sulfonic acid or other organic acid, bromide, chloride, fluoride, iodide, saccharinate, tosylate or methosulfate.

8. UV protective agent according to claim 1, characterized by the fact that the UV filter substance fraction in the vesicles referred to the total weight of the UV protective agent lies in the range from 1 to 65 wt %.

9. UV protective agent according to claim 1, characterized by the fact that the polysaccharide fraction in the vesicles referred to the total weight of the vesicles lies in the range from 1 to 85 wt %.

10. UV protective agent according to claim 1, characterized by the fact that the sum of fractions of all charge donors in the vesicles referred to the total weight of the UV protective agent lies in the range from 0.01 to 10 wt %.

11. UV protective agent according to claim 1, characterized by the fact that the hydrophobized polysaccharides from which the vesicles are constructed have a polysaccharide framework from polyglucose of polyfructose.

12. UV protective agent according to claim 1, characterized by the fact that the hydrophobized polysaccharide from which the vesicles are constructed have a polysaccharide framework from the polyglucose, which is chosen among cellulose, methyl cellulose, hydroxyethyl cellulose, amylose, amylopectin or dextrin, or a polysaccharide framework from the polyfructose inulin.

13. UV protective agent according to claim 1, characterized by the fact that the hydrophobized polysaccharide from which the vesicles are constructed are hydrophobized by the fact that the C₃-C₂₂ alkyl groups are bonded to the hydroxy groups of the polysaccharide via alkyl ether bonds or via alkyl urethane bonds or are bonded to the polysaccharide framework via a linker

14. UV protective agent according to claim 1, characterized by the fact that the at least one UV filter substance has a chemical or physical UV filter effect.

15. UV protective agent according to claim 1, characterized by the fact that the at least one UV filter substance is chosen among 3 -benzylidene camphor, 4-methylbenzylidene camphor, 1 -benzophenone, 2-benzophenone, 3-benzophenone, 4-benzophenone, 5-benzophenone, 6-benzophenone, 9-benzophenone, benzylidene camphor sulfonic acid, bis-ethylhexyloxyphenolmethoxyphenyltriazine, butylmethoxydibenzoylmethane, camphor benzalkonium methosulfate, diethylaminohydroxybenzoylhexylbenzoate, diethylhexylbutamidotriazone, disodium phenyldibenzimidazole tetrasulfonate, drometrizole trisiloxane, ethylhexyldimethyl-PABA, ethylhexylmethoxycinnamate, ethylhexylsalicylate, ethylhexyltriazone, homosalate, isoamyl-p-methoxycinnamate, methylene-bis-benzotriazolyltetramethylbutyl phenol, octocrylene, PEG-25 -PABA, phenylbenzimidazole sulfonic acid, polyacrylamidomethylbenzylidene camphor, polysilicone 15, potassium phenylbenzimidazole sulfonate, sodium mangoseedate, sodium phenylbenzimidazole sulfonate, TEA-phenylbenzimidazole sulfonate, terephthalylidene dicamphor sulfonic acid, ferulic acid, cinoxate, diisopropyl-methylcinnamate, 4-(2-β-glucopyranosiloxy)propoxy-2-hydroxybenzophenone, glyceryl ethylhexanoate dimethoxycinnamate, isopentyl trimethoxycinnamate trisiloxane.

16. UV protective agent according to claim 1, characterized by the fact that it additionally includes one or more auxiliaries.

17. UV protective agent according to claim 1, characterized by the fact that the auxiliary fraction of the vesicles referred to the total weight of the UV protective agent lies in the range of 0.01 to 10 wt %.

18. Use of a UV protective agent according to claim 1 for preparation of a cosmetic and/or pharmaceutical formulation.

19. Use according to claim 18, characterized by the fact that for production of the formulation a liposomal carrier system that penetrates the skin or hair in which at least one antioxidant active ingredient is encapsulated is additionally used.

20. Cosmetic and/or pharmaceutical formulation, which contains a UV protective agent according to claim 1 and a liposomal carrier system that penetrates the skin or hair, in which at least one antioxidant active ingredient is encapsulated.

21. Formulation according to claim 20, characterized by the fact that the liposomal carrier system consists of vesicles constructed from lipids and having a particle size from 10 to 1000 nm.

22. Formulation according to claim 20, characterized by the fact that lipids from which the liposomal vesicles are constructed are chosen among ceramides, phospholipids, glycosphingolipids and/or diacylglycosides.

23. Formulation according to claim 20, characterized by the fact that the fraction of lipids from which the liposomal vesicles are constructed, referred to the total formulation, is 1 to 20 wt %.

24. Formulation according to claim 20, characterized by the fact that the at least one antioxidant active ingredient is chosen among lipophilic and hydrophilic antioxidants, which were isolated from natural sources or were prepared chemically or by biotechnology or their combinations.

25. Formulation according to claim 20, characterized by the fact that the at least one antioxidant active ingredient is chosen among ascorbic acid, tocopherol (vitamin E), cosmetically or pharmaceutically compatible phenol compounds with one or more hydroxyl groups, derivatives of resorcinol, derivatives of hydroquinone, hydroxyl group-containing benzoic acid derivatives and cinnamic acid derivatives as well as their esters with C₂-C₁₈ alcohols or fatty alcohols, hydroxyl group-containing stilbene derivatives (for example, resveratrol) as well as ellagic acid and rosmarinic acid, chalcones, flavones, flavonols, flavanols, flavanones, flavanonols, isoflavones, anthocyans and aurones, polyphenol-rich plant extracts, antioxidant enzymes, glutathione and alkali and alkaline earth sulfites and disulfites and alkali bisulfites.

26. Formulation according to claim 20, characterized by the fact that the fraction of at least one antioxidant active ingredient referred to the entire formulation is preferably 0.01 to 20 wt %.

27. Formulation according to claim 20, characterized by the fact that it additionally includes one or more auxiliaries chosen among ethylenediamine tetraacetic acid, nitrilotriacetic acid (NTA), cosmetically or pharmaceutically compatible phosphates and/or phosphonates, salts and esters of oxalic acid and/or tartaric acid as well as N,N-bis(carboxymethyl)glutamic acid.

28. Use according to claim 18, characterized by the fact that the cosmetic and/or pharmaceutical formulation is suitable for topical application in the form of a gel, a cream, an ointment, a spray, a lotion, an aqueous or aqueous-alcoholic preparation, in which the UV protective agent and the liposomal carrier system are alternately incorporated in a carrier matrix.
